(19) United States
(12) Patent Application Publication
(10) Pub. No.: US 2016/0074657 A1
(43) Pub. Date: Mar. 17, 2016

(54) Head-wearing wireless control transcranial electrical stimulation device

(71) Applicant: ADVANCE ELECTRONIC AND MEDICAL INDUSTRIES COMPANY LIMITED, HONG KONG (CN)

(72) Inventors: Pui Tong Kwan, HONG KONG (CN); Xiang Hui KONG, HONG KONG (CN); Yu ZHAO, HONG KONG (CN)

(73) Assignee: ADVANCE ELECTRONIC AND MEDICAL INDUSTRIES COMPANY LIMITED

(21) Appl. No.: 14/949,104

(22) Filed: Nov. 23, 2015

(30) Foreign Application Priority Data
Dec. 23, 2014 (CN) 201420830404.9

(51) Int. Cl. A61N 1/36; A61N 1/20; A61N 1/372; A61N 1/04

(52) U.S. Cl. CPC A61N 1/36014; A61N 1/0476; A61N 1/20; A61N 1/37217

(57) ABSTRACT

A head-wearing wireless control transcranial electrical stimulation device includes a head-wearing part and a remote control part. An integrated circuit board is arranged within a cavity of a first end of the head-wearing part. The integrated circuit board controls a microcontroller. The microcontroller is connected with and controls a plurality of electrodes for contacting a human body through circuit modules. The microcontroller receives a control instruction from the remote control part through a Bluetooth communication terminal, and then controls the plurality of the electrodes to exert an alternating current or a direct current having preset density and frequency to a cerebral cortex. The microcontroller receives an electrode feedback signal, and sends the electrode feedback signal after being pre-processed to a display module of the remote control part to be displayed through the Bluetooth communication terminal.

### CROSS REFERENCE OF RELATED APPLICATION

The present application claims priority under 35 U.S.C. 119(a-d) to CN 201420830404.9, filed Dec. 23, 2014.

### BACKGROUND OF THE PRESENT INVENTION

### Field of Invention

The present invention relates to a field of a cranial nerve bioelectrical stimulation device, and more particularly to a head-wearing wireless control transcranial electrical stimulation device.

### Description of Related Arts

Transcranial electrical stimulation (TES) technology is a noninvasive technology which adjusts activities of nerve cells of the cerebral cortex through a weak current (0.5-2.0 mA). Since the 1990s, people have widely researched on the TES. With the continuous deepening of the research on the central nervous system and the neuroscience, people have a deeper understanding towards the TES, and meanwhile the application of the TES in treating the nerve diseases and the mental diseases, learning the motor skill and increasing the brain cognitive ability becomes possible, which lays the foundation for the further application of the TES technology in clinic and daily life.

Conventionally, the TES technology devices available on the market have following disadvantages.

Firstly, the TES technology comprises two discharging stimulation methods, respectively the transcranial direct current stimulation (TDCS) and the transcranial alternating current stimulation (TACS). Significant differences exist between the electricity generation mechanisms of the two discharging stimulation methods. Thus, during the practical application, the operator requires to use different single devices or accessories to finish the corresponding TES task, leading to the increased operation cost, the longer operation time, and the lower overall efficiency.

Secondly, the conventional TES devices available on the market have a table-shaped design, a large volume, a clumsy appearance and inconvenience in carrying. During operation, the conventional TES devices mainly adopt the alternating current (110-220V AC) to serve as the power supply, potentially risky. Moreover, the users are unable to use the conventional TES devices outdoors, because of the lack of the flexibility and the mobility.

Thirdly, in all of the conventional TES devices, the electrode plates are connected with the shielded wires. The electrode plates contact the scalp, through the conductive adhesives or the thin sponges soaked with the saline as the medium. According to the encephalic region to be stimulated, the operator finds corresponding positions on the head of the person to be stimulated, and then respectively fixes the different electrode plates on the head through the elastic adhesive tapes. Thus, during the whole TES process, because of the limitation of the length of the shielded wires, the size of the electrode plates and the firmness of the elastic adhesive tapes, the person who receives the brain electrical stimulation is required not to move casually or head movements of the person are limited. Because the brain electricity stimulation usually lasts for 15-30 min, activities of the person who receives the TES are limited, and the operator must monitor nearby, which wastes the manpower.

Fourthly, the conventional TES devices available on the market fail to detect the head skin resistance of the person who receives the TES in real time and adjust the voltage in real time according to the change of the resistance for the constant current. During the brain electricity stimulation which lasts for 15-30 min, if the poor contact, the loosening or the conductivity decrease of the conductive adhesives or the thin sponges soaked with the saline happens, the effect of the brain electrical stimulation is directly affected; however, the operator is unnoticed and fails to correct in time.

Fifthly, the conventional TES devices available on the market fail to monitor the electroencephalography (EEG) response of the brain of the stimulated person while executing the discharging stimulation. The EEG data of the person is recorded by the brain electrical device before the brain electrical stimulation, and the TES plan is set according to the results thereof. Once the TES plan is set, it is impossible to change the TES plan during the stimulation.

### SUMMARY OF THE PRESENT INVENTION

An object of the present invention is to provide a head-wearing wireless control transcranial electrical stimulation device which is portable, head-wearing, controlled and operated wirelessly, and integrates direct current (DC) and AC discharging modes.

In order to solve above technical problems, the present invention provides a head-wearing wireless control transcranial electrical stimulation device, comprising:

a head-wearing part, comprising an arch-shaped elastic damper; wherein: an integrated circuit board is arranged within a cavity of a first end of the arch-shaped elastic clamper; a microcontroller which is connected with a first Bluetooth communication terminal is arranged on the integrated circuit board; a power supply is arranged within a cavity of a second end of the elastic clamper; and the microcontroller is connected with and controls a plurality of electrodes for contacting a human body through circuit modules; and

a remote control part, comprising: a second Bluetooth communication terminal for communicating wirelessly with the head-wearing part, an operation module comprising operation buttons for editing operation information, and a display module comprising a display screen; wherein:

the microcontroller receives a control instruction from the remote control part through the first Bluetooth communication terminal, and then controls the plurality of the electrodes to exert an AC or a DC having preset density and frequency to a cerebral cortex; and, the microcontroller receives an electrode feedback signal and sends the electrode feedback signal after being pre-processed to the display module of the remote control part to be displayed through the first Bluetooth communication terminal.

Preferably, a current wave detection module, an EEG detection module and an EEG stimulation module, which are connected with the microcontroller and the electrodes, are integrated on the integrated circuit board. The current wave detection module comprises a current sensor, a first amplifier and a first analog-to-digital converter, wherein: the current sensor detects the AC or the DC flowing through the electrodes, and sends to the microcontroller after processing the AC or the DC with amplifying and analog-to-digital converting. The EEG detection module comprises an EEG sensor, a second amplifier, a filter and a second analog-to-digital converter, wherein: the EEG sensor detects an EEG signal through the electrodes, and sends to the microcontroller after respectively processing the EEG signal with amplifying, filtering and analog-to-digital converting. The EEG stimulation module comprises a digital waveform generator, a third analog-to-digital converter and a third amplifier, wherein: the microcontroller controls the digital waveform generator to generate modulation information; the modulation information is converted into an analog modulation signal through the third analog-to-digital converter and exerted to a current source; the current source outputs a current; and the current after being amplified is exerted to the cerebral cortex through the electrodes.

Further preferably, the EEG stimulation module further comprises an AC/DC switcher, wherein: the current after being amplified is firstly processed with a preset change by the AC/DC switcher, and then the AC or the DC is exerted to the cerebral cortex through the electrodes.

Further preferably, the EEG stimulation module further comprises a timer, wherein the microcontroller controls the current source to generate a constant current having a preset frequency through the timer.

Preferably, the number of the electrodes is eight.

Further preferably, the eight electrodes are divided into four pairs, and the four pairs of the electrodes are respectively preset as a DC channel, an AC channel, a grounding channel and a standby channel.

Preferably, the power supply is a charging power supply having an external charging port.

The present invention is easy to carry and operate, integrates the AC and DC stimulation discharging modes, and has a multi-mode adjustable stimulation current and stimulation frequency. The present invention is able to automatically detect a stimulation computer and an EEG response of a stimulated person, and adjust the stimulation current in real time according to a detection result.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of a head-wearing part of a head-wearing wireless control transcranial electrical stimulation device according to a preferred embodiment of the present invention.

FIG. 2 is a perspective view of a remote control part of the head-wearing wireless control transcranial electrical stimulation device according to the preferred embodiment of the present invention.

FIG. 3 is a sketch view of control circuit modules of the head-wearing part according to the preferred embodiment of the present invention.

FIG. 4 is a sketch view of circuit modules of the remote control part according to the preferred embodiment of the present invention.

In figures: 1—head-wearing part; 11—elastic damper; 12—cavity of first end of elastic clamper; 13—cavity of second end of elastic damper; 121—switch; 122—microcontroller; 123—first Bluetooth communication terminal; 124—current wave detection module; 125—EEG detection module; 126—EEG stimulation module; 131—power supply; 14—electrodes; 2—remote control part; 21—operation module; 211—operation buttons; 22—display module; 221—display screen; and 23—second Bluetooth communication terminal.

### DETAILED DESCRIPTION OF THE PREFERRED EMBODIMENT

The present invention is further illustrated with accompanying drawings and a preferred embodiment, in such a manner that one skilled in the art will better understand and implement the present invention. However, the embodiment of the present invention as shown in the drawings and described below is exemplary only and not intended to be limiting.

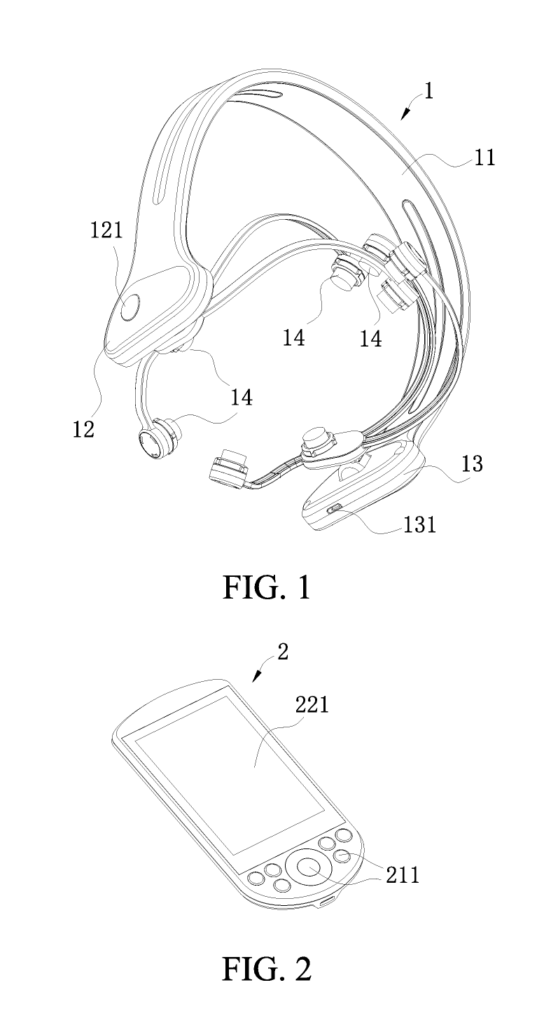

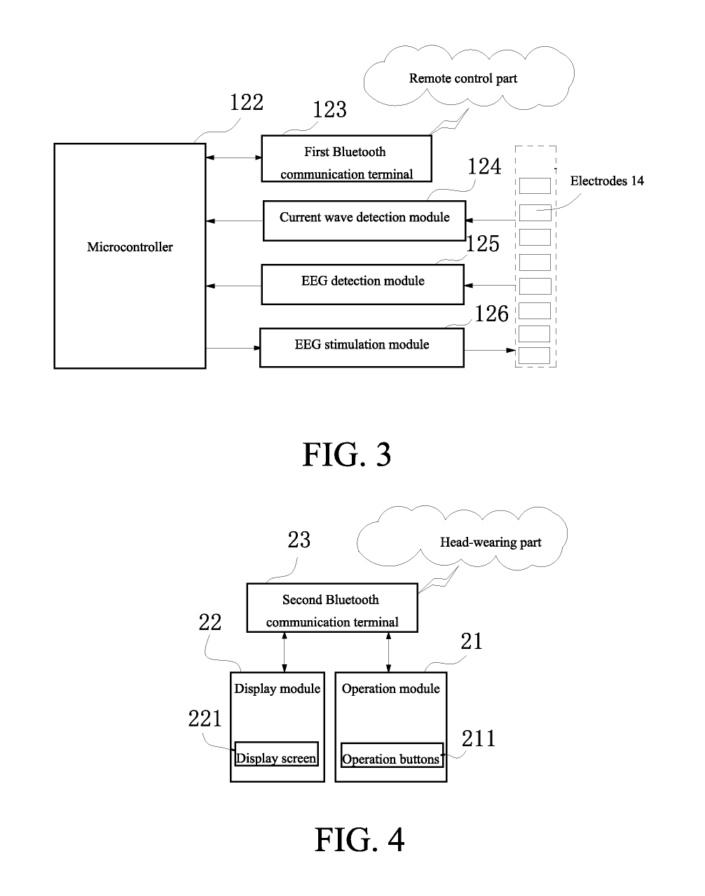

Referring to FIGS. 1-4, according to a preferred embodiment of the present invention, a head-wearing wireless control transcranial electrical stimulation device comprises a head-wearing part 1 and a remote control part 2, wherein:

the head-wearing part 1 comprises an arch-shaped elastic clamper 11; an integrated circuit board (not showed in the figures) which is started by a switch 121 is arranged within a cavity 12 of a first end of the elastic damper 11; a microcontroller 122 which is connected with a first Bluetooth communication terminal 123 is arranged on the integrated circuit board; a power supply 131 is arranged within a cavity 13 of a second end of the elastic clamper 11; and the microcontroller 122 is connected with and controls a plurality of electrodes 14 for contacting a human body through circuit modules; and

the remote control part 2 comprises: a second Bluetooth communication terminal 23 for communicating wirelessly with the head-wearing part 1, an operation module 21 comprising operation buttons 211 for editing operation information, and a display module 22 comprising a display screen 221; wherein:

the microcontroller 122 receives a control instruction from the remote control part through the first Bluetooth communication terminal 123, and then controls the plurality of the electrodes 14 to exert an AC or a DC having preset density and frequency to a cerebral cortex; and, the microcontroller 122 receives an electrode feedback signal and sends the electrode feedback signal after being pre-processed to the display module 22 of the remote control part to be displayed through the first Bluetooth communication terminal 123.

A current wave detection module 124, an EEG detection module 125 and an EEG stimulation module 126, which are connected with the microcontroller 122 and the electrodes 14, are integrated on the integrated circuit board, wherein:

the current wave detection module 124 comprises a current sensor, a first amplifier and a first analog-to-digital converter, wherein: the current sensor detects the AC or the DC flowing through the electrodes, and sends to the microcontroller after processing the AC or the DC with amplifying and analog-to-digital converting; and preferably, the current sensor is a Hall-effect sensor;

the EEG detection module 125 comprises an EEG sensor, a second amplifier, a filter and a second analog-to-digital converter, wherein: the EEG sensor detects an EEG signal through the electrodes, and sends to the microcontroller after respectively processing the EEG signal with amplifying, filtering and analog-to-digital converting; and

the EEG stimulation module 126 comprises a digital waveform generator, a third analog-to-digital converter and a third amplifier, wherein: the microcontroller controls the digital waveform generator to generate modulation information; the modulation information is converted into an analog modulation signal through the third analog-to-digital converter and exerted to a current source; the current source outputs a current, and the current after being amplified is exerted to the cerebral cortex through the electrodes.

According to the preferred embodiment of the present invention, the EEG stimulation module 126 generates the AC or the DC, and exerts to the cerebral cortex through the electrodes 14 for contacting the human body. The current wave detection module 124 obtains an actual value of the AC or the DC flowing through a brain. The microcontroller 122 adjusts a stimulation current according to detection data, and sends a result thereof to the display module 22 of the remote control part 2 through the first Bluetooth communication terminal. The display screen 221 displays current data, in such a manner that an operator is able to monitor in real time. While discharging, EEG data of the brain of a person who receives the TES is recorded in real time through the electrodes 14 for contacting the human body and the EEG detection module 125. Meanwhile, the microcontroller 122 sends the received EEG data to the display module 22 of the remote control part 2 through the first Bluetooth communication terminal 123; and the display screen 221 displays the EEG data, in such a manner that the operator monitors in real time. According to operations of the operator, parameters of the electrical stimulation are adjusted in real time.

The EEG stimulation module 126 is preferably embodied to further comprise an AC/DC switcher. The current after being amplified is firstly processed with a preset change by the AC/DC switcher, and then the AC or the DC is exerted to the cerebral cortex through the electrodes. Through the AC/DC switcher, the outputted current is changed according to requirements, and a preset AC or DC stimulation current is inputted into the cerebral cortex. The EEG stimulation module is preferably embodied to further comprise a timer, in such a manner that the microcontroller controls the current source to generate a constant current having a preset frequency through the timer for stimulating continuously.

The number and a position distribution of the electrodes 14 are mature and known to ones skilled in the art, omitted herein. It is preferably embodied that the number of the electrodes is eight. The eight electrodes are divided into four pairs, and the four pairs of the electrodes are respectively preset as a DC channel, an AC channel, a grounding channel and a standby channel. The power supply is required to supply a stable and persistent current, and the power supply is embodied to be a primary battery. It is preferably embodied that the power supply is a charging power supply having an external charging port, in such a manner that an external power supply or a storage power supply is chosen according to the requirements.

According to an AC or DC source setting of the microcontroller, the EEG stimulation module generates the DC or the AC having the current density at a certain range and the frequency at a certain range, and then exerts to the cerebral cortex. According to different time periods, control modes and detected actual currents, the microcontroller controls an intensity and the frequency of the AC or the DC flowing through the cerebral cortex through adjusting the digital waveform generator, and adjusts information of the intensity, the frequency and a shape of the AC or the DC flowing through the cerebral cortex. According to requirements of setting modes, the current wave detection module respectively sets the electrodes (1-4 channels) as DC, AC, grounding and idle. The electrodes contact the cerebral cortex of a user; electrical signals are collected to the first amplifier through all of the electrodes; amplified current signals are digitized through the first analog-to-digital converter and then sent to the microcontroller.

According to detected current data, the microcontroller calculates the intensity, the frequency and the shape of the actual current; meanwhile, the microcontroller calculates a resistance value of the cerebral cortex, sends the resistance value of the cerebral cortex to the display module of the remote control part through the first Bluetooth communication terminal, and receives user control information from the operation module of the remote control part.

Compared with the conventional technology, the present invention has following advantages.

Firstly, the conventional brain electrical stimulation device generally adopts the analog method and thus is merely able to generate the DC having a single frequency. The present invention adopts the digital EEG stimulation module which is able to change a current setting at any time and has a high accuracy. Moreover, the EEG stimulation module is able to generate an arbitrary waveform, even a complex waveform having multiple frequencies. In the meantime, the EEG stimulation module is able to generate a DC waveform or an AC waveform and has a good anti-interference performance due to the digital filter. The EEG stimulation module adopts the digital DC/AC current source and accurately controls the intensity of the current. Through the analog-to-digital converter, the AC or DC waveform having an arbitrary shape and frequency, and even the complex waveform having the multiple frequencies, are generated.

Secondly, the current wave detection module of the present invention is able to accurately detect the intensity of the current flowing through the human body. The AC/DC wave has a certain degree of distortion after flowing through the human body, and accordingly the actual value of the current flowing through the human body is inconsistent with a preset current value. The present invention collects the intensity, the shape and the frequency of the current wave flowing through the human body in real time through the first analog-to-digital converter having a high accuracy, and adjusts an output of the EEG stimulation module in real time, so as to keep the outputted current at the preset current value.

Thirdly, the present invention is able to measure a resistance of the human body. The EEG detection module detects a voltage of the human body in real-time. With an output current and a loop current of the current wave detection module, the resistance of the human body is calculated in real-time, needless of stopping the current source intermittently.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A head-wearing wireless control transcranial electrical stimulation device, comprising:
a head-wearing part, comprising an arch-shaped elastic clamper, wherein: an integrated circuit board is arranged within a cavity of a first end of said arch-shaped elastic clamper; a microcontroller which is connected with a first Bluetooth communication terminal is arranged on said integrated circuit board; a power supply is arranged within a cavity of a second end of said elastic clamper; and said microcontroller is connected with and controls a plurality of electrodes for contacting a human body through circuit modules; and
a remote control part, comprising: a second Bluetooth communication terminal for communicating wirelessly with said head-wearing part, an operation module comprising operation buttons for editing operation information, and a display module comprising a display screen; wherein:
said microcontroller receives a control instruction from said remote control part through said first Bluetooth communication terminal, and then controls said plurality of said electrodes to exert an alternating current (AC) or a direct current (DC) having preset density and frequency to a cerebral cortex; said microcontroller receives an electrode feedback signal, and sends said electrode feedback signal after being pre-processed to said display module of said remote control part to be displayed through said first Bluetooth communication terminal.

2. The head-wearing wireless control transcranial electrical stimulation device, as recited in claim 1, wherein: a current wave detection module, an electroencephalography (EEG) detection module and an EEG stimulation module, which are connected with said microcontroller and said electrodes, are integrated on said integrated circuit board; said current wave detection module comprises a current sensor, a first amplifier and a first analog-to-digital converter, wherein: said current sensor detects said AC or said DC flowing through said electrodes, and sends to said microcontroller after processing said AC or said DC with amplifying and analog-to-digital converting; said EEG detection module comprises an EEG sensor, a second amplifier, a filter and a second analog-to-digital converter, wherein: said EEG sensor detects an EEG signal through said electrodes, and sends to said microcontroller after respectively processing said EEG signal with amplifying, filtering and analog-to-digital converting; and said EEG stimulation module comprises a digital waveform generator, a third analog-to-digital converter and a third amplifier, wherein: said microcontroller controls said digital waveform generator to generate modulation information; said modulation information is converted into an analog modulation signal through said third analog-to-digital converter and exerted to a current source; said current source outputs a current; and said current after being amplified is exerted to the cerebral cortex through said electrodes.

3. The head-wearing wireless control transcranial electrical stimulation device, as recited in claim 2, wherein: said EEG stimulation module further comprises an AC/DC switcher, wherein: said current after being amplified is firstly processed with a preset change by said AC/DC switcher, and then said AC or said DC is exerted to the cerebral cortex through said electrodes.

4. The head-wearing wireless control transcranial electrical stimulation device, as recited in claim 3, wherein: said EEG stimulation module further comprises a timer; and
said microcontroller controls said current source to generate a constant current having a preset frequency through said timer.

5. The head-wearing wireless control transcranial electrical stimulation device, as recited in claim 1, wherein the number of said electrodes is eight.

6. The head-wearing wireless control transcranial electrical stimulation device, as recited in claim 2, wherein the number of said electrodes is eight.

7. The head-wearing wireless control transcranial electrical stimulation device, as recited in claim 3, wherein the number of said electrodes is eight.

8. The head-wearing wireless control transcranial electrical stimulation device, as recited in claim 4, wherein the number of said electrodes is eight.

9. The head-wearing wireless control transcranial electrical stimulation device, as recited in claim 5, wherein said eight electrodes are divided into four pairs, and said four pairs of said electrodes are respectively preset as a DC channel, an AC channel, a grounding channel and a standby channel.

10. The head-wearing wireless control transcranial electrical stimulation device, as recited in claim 6, wherein said eight electrodes are divided into four pairs, and said four pairs of said electrodes are respectively preset as a DC channel, an AC channel, a grounding channel and a standby channel.

11. The head-wearing wireless control transcranial electrical stimulation device, as recited in claim 7, wherein said eight electrodes are divided into four pairs, and said four pairs of said electrodes are respectively preset as a DC channel, an AC channel, a grounding channel and a standby channel.

12. The head-wearing wireless control transcranial electrical stimulation device, as recited in claim 8, wherein said eight electrodes are divided into four pairs, and said four pairs of said electrodes are respectively preset as a DC channel, an AC channel, a grounding channel and a standby channel.

13. The head-wearing wireless control transcranial electrical stimulation device, as recited in claim 1, wherein said power supply is a charging power supply having an external charging port.

14. The head-wearing wireless control transcranial electrical stimulation device, as recited in claim 2, wherein said power supply is a charging power supply having an external charging port.

15. The head-wearing wireless control transcranial electrical stimulation device, as recited in claim 3, wherein said power supply is a charging power supply having an external charging port.

16. The head-wearing wireless control transcranial electrical stimulation device, as recited in claim 4, wherein said power supply is a charging power supply having an external charging port.
